(19) United States
(12) Patent Application Publication
(10) Pub. No.: US 2022/0293269 A1
(43) Pub. Date: Sep. 15, 2022

(54) Computer-implemented method and electronic system for predicting a delivery time

(71) Applicant: WPMED GBR, München (DE)

(72) Inventors: Oliver PRESSMAR, München (DE); Ernst-Rainer WEISSENBACHER, München (DE)

(21) Appl. No.: 17/638,276

(22) PCT Filed: Aug. 28, 2020
(86) PCT No.: PCT/EP2020/074094

§ 371 Date: Feb. 25, 2022

(30) Foreign Application Priority Data
Aug. 29, 2019 (EP) 19194299.4

(51) Int. Cl. G16H 50/20; A61B 5/00

(52) U.S. Cl. CPC G16H 50/20; A61B 5/4343; A61B 5/7275

(57) ABSTRACT

A computer-implemented method for predicting the delivery time of a pregnant woman, including detecting at least one body parameter of the pregnant woman at multiple points in time, a calculation unit carrying out a pattern recognition with regard to the development of the at least one body parameter over time, and calculating a predicted delivery time or delivery period based on the pattern recognition. An electronic system for predicting the delivery time of a pregnant woman is also disclosed.

The present invention relates to a computer-implemented method and an electronic system for predicting the delivery time of a pregnant woman as well as to a related client-server system.

It is known in prior art to calculate a woman's monthly cycle or fertility period using electronic aids. For example, WO 2016/131630 A1 discloses a bracelet with which the skin temperature, the skin resistance, and the heart rate can be determined in order to calculate a fertility period. A self-learning algorithm is used there that can adapt to individual users. Furthermore, it is known, for example, to detect the body temperature of a woman as continuously as possible for determining the fertility period. This can be done, for example, by a sensor in the vagina. Furthermore, it is known from US 2007/0015285 A1 to determine a hormone value for ascertaining a woman's fertility period. The aforementioned self-learning algorithm adapts to the respective individual user, which is made possible by the repeating development of the monthly cycle.

It is known to predict a theoretical delivery date based on the date of the last menstrual period using the Naegele rule. For example, devices that predict ovulation based on temperature measurements can provide an indication of the theoretical delivery date at the beginning of a pregnancy, as disclosed for example, in JP 2009-045155 and JP 2007-068839. However, the calculation of the delivery date is rather imprecise and does not take into account the actual development of the pregnancy.

EP 0 648 335 B1 discloses that in pregnancies with a risk of premature labor or in transferred pregnancies, a test kit can be used to determine the presence of a hormone in the pregnant woman's vagina, which can be used to determine whether delivery is imminent. Furthermore, medical studies have shown that a change in body temperature occurs in pregnant women in the days leading up to delivery. However, the relevant detection and evaluation is relatively complicated for pregnant women and is overlaid by other factors so that it is not possible to reliably predict the delivery time without medical support.

The object of the present invention is to provide a pregnant woman with a method and an electronic system with which the delivery time can be predicted reliably and in a simpler manner.

The invention provides a computer-implemented method for predicting the delivery time of a pregnant woman in which at least one body parameter of a pregnant woman is detected at multiple points in time, pattern recognition is carried out with regard to the development of the at least one body parameter over time, and a predicted delivery time or delivery period is calculated based on the pattern recognition.

In particular, the delivery time or delivery period is reproduced visually, acoustically, or haptically, preferably displayed visually. In addition to displaying the delivery time, a possible time deviation may be displayed, which reflects the calculation accuracy of the prediction. Alternatively, a predicted delivery period may be specified, the length of which reflects the calculation time. In particular, a probability may be displayed or selected according to which the delivery may be expected to take place within the aforementioned time deviation or within the aforementioned delivery period. In particular, with increasing body parameter detection, an ever-increasing probability with regard to the prediction may be displayed for the same time deviation or for the same predicted delivery period, or an ever-decreasing time deviation or an ever-decreasing predicted delivery period for the same probability with regard to the prediction. Due to the uncertainty of the prediction, a predicted delivery time is in particular the statistical midpoint of a predicted delivery period.

In contrast to a woman's menstrual cycle, the development of a pregnancy is not a cyclical process to which a self-learning algorithm may be adapted. The method according to the invention enables the delivery time to be predicted without recourse to previous pregnancies by the same woman. The pattern recognition is instead carried out by analyzing the development of at least one significant body parameter over time. At least one of the body parameters evaluated is advantageously detected at time intervals of less than six hours, three hours, one hour, 30 minutes, 15 minutes, 5 minutes, 1 minute, 30 seconds, 15 seconds, or even less than 1 second. The method is advantageously carried out at least or only in the last six, five, four or three weeks prior to the conventionally predicted delivery time.

The invention further provides a computer-implemented method for predicting the delivery time of a pregnant woman in which at least two, but advantageously five, more advantageously more than ten, and still more advantageously more than twenty body parameter of a pregnant woman are detected at just one point in time, and pattern recognition with regard to the correlation of the detected body parameters is carried out by a calculation unit, where a predicted delivery time or delivery period is calculated based on the pattern recognition. The detected body parameters may be the body parameters already discussed above, where their correlation may also provide information about the development of the pregnancy over time with regard to the delivery time. Correlations with regard to the precursors of hormones or their breakdown products may provide information with regard to the development over time.

It is possible to optimize a set of parameters for pattern recognition, in particular a set of parameters for an artificial neural network, by detecting a plurality of body parameters together with the actual delivery time.

The at least one body parameter may be in particular a substance concentration in a body fluid of the pregnant woman, in particular in the urine, blood, saliva, or vaginal secretion. In particular, the substance concentration of at least one of the following substances is detected as a body parameter: corticotropin-releasing hormone (CRH), adrenocorticotropic hormone, cortisol, progesterone, estrogen, inflammatory cytokines, prostaglandin, glucocorticoids, dehydroepiandrosterone, C-reactive protein, leukocytes, and/or oxytocin. In particular, a drop in the progesterone concentration is a clear indication of imminent delivery. In particular, the progesterone-estrogen ratio shifts towards an overbalance of estrogen. The progesterone drop may also be detected indirectly by way of a different substance concentration. For example, detecting an increase in the estrogen concentration or the estrogen precursor dehydroepiandrosterone (DHEA) is an alternative indicator of a drop in the progesterone concentration. In particular, by detecting concentrations of different substances, which represent, for example, precursors to one another, it is possible to detect a development of a substance concentration over time with only one measurement at one point in time.

Furthermore, at least one of the body parameters may be a vital parameter (in particular heart rate, blood flow, body temperature, breathing frequency, electrical resistance of the skin, and/or blood pressure). Detecting these values is comparatively simple, in particular using a wearable device such as a bracelet, and may therefore take place at short time intervals and in an automated manner. The development of the body temperature is presently of particular interest. In particular, a drop in temperature may arise on the days prior to labor, combined with a rise in temperature just prior to labor. These temperature developments are known in the medical and veterinary field to predict the delivery time, but similar developments may also occur earlier in the course of pregnancy, so that, without medical expertise, a false prediction regarding a possibly imminent delivery may be made which may lead to uncertainty on the part of the respective pregnant woman. For this reason, the body temperature must presently be detected precisely and in detail, and a correspondingly precise pattern recognition as is made possible by the computer-implemented method according to the invention.

In a preferred embodiment, the body temperature and the progesterone concentration are detected as body parameters. In other preferred embodiments, the concentrations of estrogen, a cytokine, and/or corticotropin-releasing hormone (CRH) are also detected as body parameters.

In one embodiment, the computer-implemented method may comprise detecting a manual input, specifically a body parameter additionally determined, and calculating the predicted time or period of delivery while taking into account the body parameter additionally determined. The body parameter additionally determined may be in particular a contraction frequency and/or at least one parameter from a cardiotocography. The body parameter additionally determined is advantageously a body parameter that is detected or that may be additionally detected during a prenatal check-up. If the body parameter additionally determined is available in the computer-implemented method in addition to the detected body parameters at several points in time, then the calculation of the predicted delivery time or delivery period may be adjusted or detected in a more precise manner. In one embodiment of the method, the body parameter additionally determined is entered manually by medical staff as part of a prenatal check-up, whereupon the predicted delivery time or delivery period is updated in an automated manner.

Advantageously, at least two different body parameters are detected, where the pattern recognition comprises an identification of correlations in the developments of these at least two body parameters. In particular, at least one body parameter that is easy to detect, for example, a vital parameter, may be detected at short time intervals, for example, at least once an hour, supported by the detection of at least one body parameter that is more difficult to detect, for example, a substance concentration in a body fluid, at longer intervals, for example, at least every day or two. This combination significantly increases the accuracy of the prediction with a slight increase in effort.

In one embodiment, the predicted delivery time or delivery period is displayed at least three days prior to the predicted delivery time or delivery period, respectively. In further preferred embodiments of the method, the displaying takes place at least five days, at least ten days, at least two weeks, or at least four weeks prior to the predicted delivery time. The better the database for the pattern recognition, i.e. the number of detected body parameters, their significance, their number per unit of time, as well as the degree of optimization of the pattern recognition of the set of parameters, the earlier the displaying may take place.

In one embodiment, the selection of which body parameter or parameters is/are detected is done depending on the time remaining until the predicted delivery time or delivery period. Some body parameters, such as substance concentrations in a bodily fluid, in particular hormone values, may enable a more long-term prediction, whereas other body parameters, for example, vital parameters, in particular body temperature or heart rate, enable a more short-term prediction with regard to the delivery time or delivery period. It is then possible to primarily or exclusively detect the long-term predictive body parameters for a longer remaining time until the predicted delivery time or delivery period, and the short-term predictive body parameters for a shorter remaining time until the predicted delivery time or delivery period.

In one embodiment, the actual delivery time is detected in an automated manner or by manual input. Based on this, a plurality of data sets of detected body parameters is evaluated in their development over time up to the actual delivery time in order to recognize patterns, the patterns are stored and made available for pattern recognition. In particular, a set of parameters for pattern recognition is stored and made available. The actual delivery time may be detected in an automated manner, for example, triggered by vital parameters detected that are characteristic of a delivery, or by manual input by the user or by third parties, in particular medical staff. The pattern recognition carried out according to the invention is preferably carried out by artificial neural networks. The set of parameters for the pattern recognition may be, for example, the set of parameters of an artificial neural network which is obtained by optimization using an optimization algorithm based on the detected body parameters as input data and the detected actual times of delivery as output data. Artificial neural networks make it possible, in particular in the presence of a large number of data sets, to determine correlations that could not be determined using deductive methods or conventional data analysis, and significantly improve the prediction accuracy. In one embodiment, a single-layer, non-recurrent artificial neural network is employed. In other embodiments, multi-layer, non-recurrent networks with one or more hidden layers of artificial neurons in front of the output layer may also be provided.

In other embodiments, it is possible to use other artificial intelligence or machine learning algorithms.

In one embodiment, the pattern evaluation may be weighted as a function of the specific parameters by storing specific parameters relating to the pregnant woman or her environment. In particular, an input layer of an artificial neural network may be optimized with regard to the specific parameters, and the inner layers with regard to the body parameters. By optimizing the topology of the artificial neural network in this manner, significant improvements in terms of pattern recognition may be achieved.

The invention furthermore provides an electronic system for predicting the delivery time of a pregnant woman which comprises at least one sensor for detecting at least one variable body parameter, and a calculation unit which is configured to determine a predicted delivery time or delivery period based on pattern recognition in the development over time of the at least one detected body parameter. Advantageously, the device is configured to display the predicted delivery time or delivery period only from a predetermined accuracy of the prediction.

The invention furthermore provides an electronic system for predicting the delivery time of a pregnant woman with at least one sensor for detecting at least two variable body parameters, and a calculation unit which is configured to determine a predicted delivery time or delivery period based on pattern recognition of the at least one detected body parameter. At least five, at least ten, at least twenty, or at least fifty body parameters are advantageously used as a basis for the pattern recognition.

Advantageously, the electronic system comprises a wearable device, in particular a bracelet, where the at least one sensor is arranged in the wearable device, and the at least one sensor is a heart rate sensor, blood flow sensor, temperature sensor, skin resistance sensor, and/or acceleration sensor. By detecting the perfusion using the blood flow sensor, the estrogen or progesterone concentration in the blood may be determined indirectly, since these hormones change the blood flow in dependence of their concentration.

In particular, the electronic system comprises an evaluation unit in which the at least one sensor is arranged in the form of a biosensor for a body fluid. The evaluation unit may be, for example, a pin-like device that may be arranged in a body fluid that has been discharged or in a body opening.

The electronic system advantageously comprises an input field via which a body parameter measured may be recorded by manual input. In particular, the input field may be arranged on the bracelet of the electronic system.

In particular, the electronic system may comprise a smartphone, alternatively or additionally also another computer, for example, a laptop computer or desktop computer in which the calculation unit and/or the input field is/are provided.

In one embodiment, the at least one sensor or the calculation unit may be connected to a server by way of a communication unit, where the at least one sensor or the calculation unit is configured to transmit at least some of the detected body parameters to the server by way of the communication unit, and the server to obtain the result of the pattern recognition regarding the transmitted data. The server may comprise the calculation unit. The set of parameters for the pattern recognition may be optimized with each data set transmitted to the server or at given points in time on the basis of the newly received data sets. Furthermore, it is possible to use this optimized set of parameters for each pattern recognition and therefore to always ensure the best possible results. Additionally or alternatively, the electronic system may be operated in an offline mode of operation. For this purpose, the calculation unit comprises a memory in which a set of parameters for pattern recognition is stored, where the calculation unit is configured to calculate the predicted delivery time or delivery period based on the measured body parameters using the stored set of parameters. The parameter data set may be updated by transferring data from a server.

In particular, the set of parameters is a set of parameters for an artificial neural network, stored patterns relating to body parameters, or information relating to correlations between different body parameters.

The invention further provides a client-server system for predicting the delivery time of a pregnant woman, where the client is formed by an electronic system as described above or is configured to carry out a computer-implemented method as described above, and where a server is provided which is configured to compare the at least some transmitted body parameters detected by the client with patterns and correlations stored on the server, and to use this to calculate the predicted delivery time or delivery period.

In a preferred embodiment of the computer-implemented method, a body temperature of the pregnant woman is detected at least twice a day, advantageously in the morning and in the evening. In particular, the body temperature of a pregnant woman follows a circadian rhythm having a higher body temperature in the morning and a lower body temperature in the evening. If, in particular, the temperature in a morning measurement is lower than in the previous evening measurement of the body temperature, then this may be significant for the pattern recognition and calculation of the predicted delivery time or delivery period. Such a change in the circadian rhythm is particularly significant in a range from five, from four, or from three days prior to a delivery time predicted by other patterns. Furthermore, it is possible to determine the degree of cellular inflammatory reactions as a body parameter by way of a substance concentration in a body fluid, which may also be meaningful in terms of the prediction of the delivery time.

In addition, a progesterone value of the pregnant woman may be detected at least every three days, at least every two days, or once a day. A pattern recognition with regard to the temperature and progesterone profile may then be used to calculate the predicted delivery time or delivery period, advantageously at least three days prior to labor. A drop in the progesterone concentration leads to a reduction in body temperature due to the elimination of the hyperthermic effect of progesterone. This occurs in particular in the 48, 36, or 24 hours prior to labor. In particular, a drop in progesterone from 6 ng per millimeter to 2 ng per millimeter is significant. The computer-implemented method may then be configured to determine a reduction in the progesterone concentration to below 50%, below 40%, below 30% or below 20%, starting from an initial value, as being significant for the pattern recognition. The drop in progesterone increases the effect of estrogens and leads to an increase in the contractility of the smooth muscles of the uterus.

As additional information, a theoretical due date may be calculated on the basis of the woman's last cycle according to the Naegele rule and taken into account when calculating the predicted delivery time or delivery period.

In addition, measured variables relating to the unborn child detected by ultrasound biometry, such as the transverse diameter of the thorax (MAD), the crown-rump length (SSL) or the biparietal diameters (BPD) of the unborn child, i.e. body parameters of the unborn child, may be taken into account in the pattern recognition.

In particular, an evaluation unit may be provided which is configured to receive test strips with which substance concentrations in a body fluid may be detected. The evaluation unit may comprise, in particular, a display and may store or evaluate measurement values and transmit them to the pattern recognition. It is also possible that the biosensor is a short-term implant that is inserted, for example, into the vagina and there may determine the concentrations of at least one hormone, in particular the progesterone concentration, but preferably the concentrations of at least 3, 5, or 10 hormones, or the body temperature.

The invention shall be explained hereafter using an exemplary embodiment with reference to the following figures:

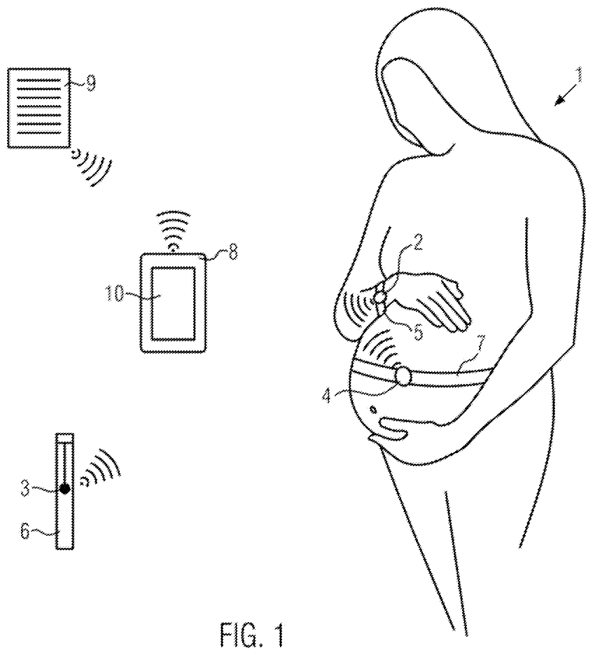

FIG. 1 shows an embodiment of an electronic system according to the invention with a pregnant woman,

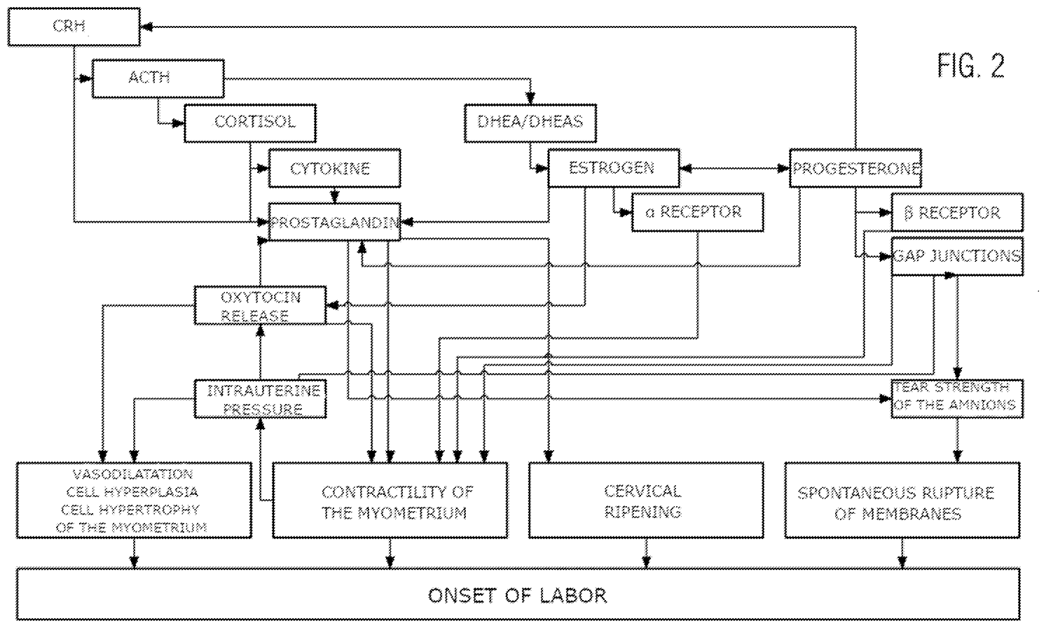

FIG. 2 shows the chronological sequence of the changes in body parameters that may be used as a basis for pattern recognition for predicting the delivery time or delivery period in embodiments of the method and the system according to the invention.

FIG. 1 shows a pregnant woman 1 with an embodiment of an electronic system for predicting the delivery time. The electronic system comprises several sensors 2, 3, 4 with which body parameters of the pregnant woman may be detected. In particular, this is a sensor 2 in a bracelet 5 for detecting vital parameters, in particular heart rate, blood flow, body temperature, electrical resistance of the skin, and/or blood pressure.

A further sensor 3 is arranged in an evaluation unit 6. This sensor may be, for example, a photo sensor which evaluates test strips inserted into the evaluation unit. The test strips in particular may have been exposed to a body fluid of the pregnant woman and may have different local color changes with regard to substance concentrations which may then be detected by the photo sensor so that one substance concentration in the body fluid or several substance concentrations may be determined. Alternatively, sensor 3 may be a biosensor with which substance concentrations in a body fluid of the pregnant woman may be determined directly.

Further sensor 4 may be arranged in a waist belt 7. It may be used to determine further vital parameters, such as breathing frequency or labor frequency, or body parameters of the unborn child.

Bracelet 5, evaluation unit 6, and/or waist belt 7 may each be provided with a radio module with which the detected body parameters may be transmitted. In particular, the detected body parameters may be transmitted to a smartphone 8 and combined there. Alternatively, however, it is also possible for the data to be received and combined in one or all of bracelet 5, evaluation unit 6, or waist belt 7. Smartphone 8, bracelet 5, evaluation unit 6, and/or waist belt 7 comprise a calculation unit which is configured to determine a predicted delivery time or delivery period based on pattern recognition from one or more detected body parameters. Alternatively, the body parameters determined may be transmitted to a server 9 and the pattern recognition can be carried out on the server. Furthermore, smartphone 8, bracelet 5, evaluation unit 6, and/or waist belt 7 may comprise a display unit 10 on which the predicted delivery time or delivery period is displayed. Alternatively or additionally, a notification may be given, for example, by audio signal or vibration of smartphone 8, waist belt 7, evaluation unit 6, and/or bracelet 5.

FIG. 2 shows the cascading sequence leading up to the start of labor, with relevant body parameters being indicated. A prediction according to the invention of the delivery time or delivery period may be carried out by detecting these body parameters at least in part, at least in the sections of the sequence in which these body parameters are relevant, in particular show significant changes. An organized equilibrium (OG) between pregnancy-maintaining and labor-promoting body parameters arises during pregnancy. Auto-, para- and endocrine processes play a role there (HH-NNR of the mother or the fetus and from the fetomaternal boundary region). Further relevant body parameters are the neurotransmitter CRH, the steroid hormones progesterone and estrogen, the glucocorticoids, oxytocin, as well as the prostaglandins, cytokines, etc.

Labor-promoting hormones are in particular CRH, ACTH, DHEA, estrogen, estradiol, cytokines IL-1β, IL-6, IL-8, prostaglandin PGE₂ PGF₂, and oxytocin.

Labor-inhibiting hormones and mediators are in particular progesterone and prostacyclin PGI₂.

Changes in the fetal membranes (ruptured membranes), the myometrium (contraction) and the cervix (ripening) are relevant for triggering labor. The processes that lead to these changes shall be described below.

The basic process of labor induction follows a cascading process that may lead to a shift in the organized balance (OG) and may thus lead to delivery that is early, on-time or after the date. The closer the delivery, the greater the changes in the tissue (decidua, myometrium, cervix), which are mainly an expression of inflammation or infection with changes in the microbiological and macrobiological region and may be determined by way of body parameters indicative of inflammation or infection.

The following body parameters should advantageously be taken into account:

I. Release of CRH and maturation of fetal HH-NNR

II. Inflammation or infections, respectively

III. Shift in the progesterone-estrogen ratio

IV. Increased prostaglandin synthesis

V. Increase in intrauterine pressure

VI. Release of oxytocin

In the microbiological field, qualitative and quantitative changes in the spectrum of germs (bacteria, viruses, fungi, etc.) may occur. Likewise, the metabolism of the germs, e.g. the protease formation—with an effect on the arachidonic and citric acid cycle—has a significant influence. In the macrobiological field—i.e. in humans—the processes taking place in close connection with the microbiological field trigger an immunological cascade with cell-bound and humoral responses (cytokines, etc.). This may be determined by way of suitable body parameters. In detail:

I. Release of CRH and Maturation of Fetal HH-NNR

During pregnancy, corticotropin-releasing hormone (CRH) is produced in tissues of the fetomaternal boundary region, the placenta, the chorion, the cells of the amnion, and the decidua. In the fetus, this leads to maturation of the fetal HH-NMR and production of adrenocorticotropic hormone (ACTH) in the fetal anterior pituitary gland. This in turn leads to increased release of cortisol, the concentration of which may be determined as a body parameter. Furthermore, the increased cortisol level activates inflammatory cells and thereby releases more inflammatory cytokines and prostaglandins, the concentrations of which may be determined as body parameters. CRH also has a prostaglandin-enhancing effect and, at higher levels, leads to vasodilatation and an increase in myometrial contractility.

II. Inflammation or Infections, Respectively

The cortisol level increased according to the aforementioned mechanisms or as a side access due to an infection in the body (e.g. periodontal disease or the amnion due to pathogenic or potentially pathogenic germs) leads to local inflammation in infections and generally to a release of cytokines such as −1, −6, −8, −10, −12, etc. or tumor necrosis factors and factors stimulating granulocyte colonies. An increase in the number of leukocytes is one of the consequences. Together with the inflammation-induced stimulation of arachidonic acid metabolism, prostaglandin production is increased, which may be determined in the context of body parameter detection. This leads to cervical ripening and (premature) labor. The inflammation at the egg pole as part of the amnion infection also leads to damage to the egg membranes and increases the risk of ruptured membranes.

III. Shift in the Progesterone-Estrogen Ratio

The production of ACTH increases the release of the estrogen precursor dehydroepiandrosterone (DHEA). Glucocorticoids may also be indicative of the onset of labor. DHEAS increases in parallel with estrogen towards the end of pregnancy. This shifts the progesterone-estrogen ratio towards an overbalance of estrogen. This has two implications. Estrogen has a contraction-promoting effect on the α-receptors and has a positive influence on the prostaglandin and oxytocin receptors in the myometrium. Progesterone, on the other hand, activates the β-receptors, which induces a relaxing effect. Progesterone has a suppressive effect on gap junction synthesis as well as on CRH production. There is also a drop in progesterone production in the placenta towards the end of the pregnancy, which in turn is another building block that promotes delivery and also has an effect on thermogenesis.

IV. Increased Prostaglandin Synthesis

Progesterone is converted to estrogen in the placenta and stimulates the production of prostaglandin F_{2α} (PGF_{2α}) in the myometrium. The prostaglandins induce contractions in the myometrium, thereby increasing the intrauterine pressure and causing vasodilation of the myometrium. They also influence cervical ripening and spontaneous rupture of membranes.

V. Increase in Intrauterine Pressure

The intrauterine stretching stimulus leads to cell hyperplasia and cell hypertrophy in the myometrium and to an increase in the expression of contraction-associated proteins (GAP junctions connexin H3) as well as to impairment of the tear strength of the amnions.

VI. Release of Oxytocin

The dilation of the cervix leads to the release of oxytocin from the maternal posterior pituitary gland. The oxytocin, in turn, stimulates prostaglandin synthesis and induces additional contractions of the myometrium to fully expel the fetus from the uterus.

In particular, the following substances or their concentrations may be detected as physical parameters: CRH, ACTH, cortisol, cytokines, prostaglandins, DHEA/DHEAS, estrogen, a receptor, progesterone, β receptor, and/or oxytocin. With the onset of labor, the muscle cells of the uterus have gap junctions whereby they are connected to form a syncytium and may contract synchronously.

The processes shown in FIG. 2 lead to vasodilatation, cell hyperplasia, cell hypertrophy of the myometrium, contractility of the myometrium, cervical ripening, and/or spontaneous rupture of membranes. These events are in turn direct precursors to the onset of labor.

There are also side accesses in the labor cascade, which may also be detected by determining body parameters:

A. Stress or Hypoxic-Ischemic Lesions of the Placenta

Chronic maternal stress may prematurely activate the fetal HH-NNR axis in that CRH release is activated and cytokines are released. As the pregnancy progresses, desensitization to an increased CRH level may arise and the stress trauma may thus be reduced.

B. Effects of Circadian Rhythm on Interleukin IL-β and TNF

There is increased activity of the myometrium in the second half of the night and in the early morning, as well as an increase in progesterone and DHEAS levels, as well as maternal estradiol and progesterone fluctuations.

The aforementioned changes in the pregnant woman's body are reflected in her body parameters, either directly, for example, by the substance concentration of a hormone, or indirectly, for example, by the change in a vital parameter. An inflammatory reaction may lead to increased body temperature and/or altered heart rate values. The detection of at least one body parameter over time and/or of several different body parameters and a related pattern recognition, in particular with regard to correlations between the body parameters, may therefore enable exact prediction of the delivery time or delivery period.

1. A computer-implemented method for predicting the delivery time of a pregnant woman, comprising the steps of:
detecting at least two body parameters of said pregnant woman (1) at multiple points in time;
carrying out a pattern recognition by a calculation unit with regard to the development of said at least two body parameters over time; and
calculating a predicted delivery time or delivery period by the calculation unit based on said pattern recognition;
wherein at least one of said at least two body parameters is a substance concentration in a body fluid of said pregnant woman; and
wherein at least another of said at least two body parameters is a vital parameter.

2. The computer-implemented method according to claim 1, wherein the substance concentration is of at least one of the following substances: corticotropin-releasing hormone, adrenocorticotropic hormone, cortisol, progesterone, estrogen, inflammatory cytokines, prostaglandin, glucocorticoids, dehydroepiandrosterone, C-reactive protein, leukocytes, and/or oxytocin.

3. The computer-implemented method according to claim 1, wherein the vital parameter is, a heart rate, a blood flow, a body temperature, a breathing frequency, an electrical resistance of the skin, and/or a blood pressure, of the pregnant woman (1).

4. The computer-implemented method according to claim 1, further comprising detecting a manual input of an additionally determined body parameter wherein the additionally determined body parameter is a contraction frequency and/or at least one parameter from a cardiotocography, and wherein calculating a predicted delivery time or delivery period includes taking into account said additionally determined body parameter.

5. The computer-implemented method according to claim 1, wherein said pattern recognition comprises a recognition of correlations in the developments of the at least two body parameters.

6. The computer-implemented method according to claim 1, further comprising displaying the predicted delivery time or delivery period with a display at least three days prior to the predicted delivery time.

7. The computer-implemented method according to claim 1, further comprising selecting a body parameter or parameters for detection in dependence on a time remaining until the predicted delivery time or delivery period.

8. The computer-implemented method according to claim 1, further comprising detecting an actual delivery time in an automated manner or by manual input, and evaluating a plurality of data sets of detected body parameters in their development over time towards the actual delivery time for recognizing patterns, storing said patterns, and providing said patterns for pattern recognition.

9. The computer-implemented method according to claim 1, further comprising storing specific parameters relating to said pregnant woman or her environment, and weighting of said pattern recognition in dependence of said specific parameters.

10. An electronic system for predicting the delivery time of a pregnant woman (1), comprising:
at least one sensor (2, 3, 4) configured for detecting at least two variable body parameter and
a calculation unit configured to determine a predicted delivery time or delivery period on a basis of a pattern recognition in a development over time of said at least two variable body parameters detected by the at least one sensor;
wherein at least one of said at least two variable body parameters is a substance concentration in a body fluid of said pregnant woman; and
wherein at least another of said at least two variable body parameters is a vital parameter.

11. The electronic system according to claim 10, further comprising a wearable device (4, 5, 6) comprising a bracelet (5), wherein said at least one sensor (2, 3, 4) is arranged in said wearable device (4, 5, 6), and wherein said at least one sensor (2, 3, 4) is a heart rate sensor, a blood flow sensor, a temperature sensor, a skin resistance sensor, and/or an acceleration sensor.

12. The electronic system according to claim 10, further comprising an evaluation unit (6) in which said at least one sensor (3) is arranged, wherein the at least one sensor is a biosensor for a body fluid.

13. The electronic system according to claim 10, further comprising an input field (10) via which a measured body parameter is recordable by manual input.

14. The electronic system according to claim 10, wherein said at least one sensor (2, 3, 4) is connectable to a server (9) by way of a communication unit which is configured to transmit data corresponding to at least some of said at least two variable body parameters detected by said at least one sensor to said server (9) and to obtain from said server (9) the result of said pattern recognition regarding said transmitted data.

15. A client-server system for predicting the delivery time of a pregnant woman, comprising:
a client formed by the electronic system according to claim 10, and a server (9) configured to compare data corresponding to at least some of said at least two variable body parameters detected by the client with patterns and correlations stored on said server (9) and to use said comparison to calculate the predicted delivery time or delivery period.

16. A computer-implemented method according to claim 1, wherein the substance concentration is of progesterone and the vital parameter is a body temperature of the pregnant woman (1).

17. A computer-implemented method according to claim 5, wherein the substance concentration is of progesterone and the vital parameter is a body temperature of the pregnant woman (1).

18. A computer-implemented method according to claim 1, wherein the body fluid is saliva.

19. A computer-implemented method according to claim 16, wherein the body fluid is saliva.
